(19) United States
(12) Patent Application Publication
(10) Pub. No.: US 2023/0000319 A1
(43) Pub. Date: Jan. 5, 2023

(54) METHOD AND APPARATUS FOR BIOMETRIC TISSUE IMAGING

(71) Applicant: Optosurgical, LLC, Columbia, MD (US)

(72) Inventors: Jaepyeong CHA, Fulton, MD (US); Hun Chan LEE, Brookline, MA (US)

(73) Assignee: Optosurgical, LLC, Columbia, MD (US)

(21) Appl. No.: 17/854,084

(22) Filed: Jun. 30, 2022

Related U.S. Application Data
(60) Provisional application No. 63/217,156, filed on Jun. 30, 2021.

(51) Int. Cl. A61B 1/00; A61B 1/05; A61B 1/06

(52) U.S. Cl. CPC A61B 1/00124; A61B 1/053; A61B 1/0638; A61B 1/0646; A61B 1/00126

(57) ABSTRACT

Provided are medical imaging apparatuses that comprising an optical connector, a coupler configured to releasably couple to a first portion of the optical connector and a camera configured to releasably couple to a second portion of the optical connector, wherein the coupler comprises a first portion to which light is incident from the optical connector, and a second portion in which the light passes through an inside of the coupler and is emitted, wherein the first portion is tilted with a predetermined angle with respect to the second portion.

### CROSS-REFERENCE TO RELATED APPLICATIONS

This application claims priority to and the benefit of, U.S. Provisional Application Ser. No. 63/217,156 filed Jun. 30, 2021, entitled “METHOD AND APPARATUS FOR BIOMETRIC TISSUE IMAGING”, which is incorporated herein in the entirety by reference.

### A. Technical Field

The present disclosure relates to a method and apparatus for biometric tissue imaging, more particularly, to a method and apparatus capable of identifying a tissue within a subject's body in real time.

### B. Description of the Related Art

Medical imaging technology is being used to capture images or video data of internal anatomical or physiological features of a subject or patient during medical or surgical procedures. The images or video data captured may be processed and manipulated to provide medical practitioners (e.g., surgeons, medical operator, technicians, etc.) with a visualization of internal structures or processes within a patient or subject.

Images or video data of internal anatomical or physiological features by a medical imaging apparatus (e.g., scope assembly) may be limited and often fail to provide complex anatomy or critical structures beneath the tissue surface. As a result, incomplete or incorrect identification of the target site may be dangerous and lead to unintended tissue damage during surgical procedures.

Meanwhile, sterile barrier assemblies such as surgical drapes are known for establishing barriers between surgical components during surgery. For instance, a surgical drape may be used to provide a sterile barrier with an optically clear window between a scope and an optical connector including a camera. In surgery, the scope is treated as being sterile, while the optical connector is nonsterile. The surgical drape creates a barrier between the scope and the optical connector to prevent contamination of a sterile field in which the optical connector is operating.

The surgical drape functionally prevents contamination of surgical components, but when the light emitted from the light source to recognize the target, passes through the surgical drape during surgical procedures, reflected light is generated from the window installed inside the surgical drape causes the practitioners to be misunderstood and confused in recognizing the target.

Therefore, there is a need in the art for addressing one or more of these deficiencies.

### SUMMARY

The present disclosure addresses at least the above-mentioned shortcomings medical imaging systems. In one aspect, the present disclosure provides a coupler that can be compatible with one or more medical imaging devices (e.g., an optical connector). In another aspect, the present disclosure provides a medical imaging apparatus that can be compatible with a surgical drape (e.g., a sterile drape assembly) during surgical procedures. In a further aspect, the present disclosure provides a sterile drape assembly that can be compatible with one or more medical imaging devices (e.g., an optical connector). In an additional aspect, the present disclosure provides an optical connector that can be compatible with a surgical drape (e.g., a sterile drape assembly) during surgical procedures.

One aspect of the present disclosure provides a coupler for medical imaging comprising: an adapting unit including an opening therein; a connecting unit configured to couple to the adapting unit; a securing unit configured to couple to the connecting unit; and a covering unit configured to couple to the securing unit, wherein each of the connecting unit, the securing unit and the covering unit has a hollow in spatially communication with the opening of the adapting unit and one side of the adapting unit is inclined with a predetermined angle with respect to the opposite side of the adapting unit.

In some embodiments, the adapting unit may comprise a fixing part having a first opening for passing light and an angle adjustment part configured to be integral with the fixing part, having a second opening in spatially communication with the first opening of the fixing part.

In some embodiments, a diameter of the second opening may be larger than the diameter of the first opening

In some embodiments, the connecting unit may comprise a plate with a groove and a spring member inserted into the groove.

In some embodiments, the securing unit may comprise a baseplate, a guide protrusion protruding from one side of the baseplate, and a grip plate extending parallel to the guide protrusion from an edge portion of the baseplate.

In some embodiments, the covering unit may comprise a cover plate and a flange extending substantially perpendicular to a surface of the cover plate.

In some embodiments, a central axis of the opening may be tilted with a given angle with respect to a central axis of the hollow.

Another aspect of the present disclosure provides a medical imaging apparatus comprising: an optical connector; a coupler configured to releasably couple to a first portion of the optical connector; and a camera configured to releasably couple to a second portion of the optical connector, wherein the coupler comprises a first portion to which light is incident from the optical connector, and a second portion in which the light passes through an inside of the coupler and is emitted, wherein the first portion is tilted with a predetermined angle with respect to the second portion.

In some embodiments, a wavelength of the light may range from 785 nm to 830 nm.

In some embodiments, the optical connector may comprise a casing disposed along an optical path of the light, that includes a beam splitter and a beam dump located on an opposite portion of a surface of the beam splitter on which the light is incident in the casing.

In some embodiments, the camera may comprise a first image sensor and a second image sensor for respectively sensing a near-infrared light and a visible light emitted from a target irradiated with the light.

In some embodiments, the second portion of the coupler may be configured to releasably couple to a sterile adapter using a quick release mechanism.

A further aspect of the present disclosure provides a medical imaging system comprising: an optical connector; a sterile adapter; and a coupler having a first portion to releasably couple to a first side of the optical connector and a second portion to releasably couple to the sterile adapter, wherein the first portion of the coupler is tilted with a predetermined angle with respect to the second portion of the coupler.

In some embodiments, the sterile adapter may comprise a window positioned within the sterile adapter so that light incident from the coupler is not retroreflected at a surface of the window.

In some embodiments, the medical imaging system may further comprise a sterile drape connected to the sterile adapter using a locking ring.

In some embodiments, the medical imaging system may further comprise a camera configured to releasably couple to a second side of the optical connector, wherein the camera comprises a first image sensor and a second image sensor for respectively sensing a near-infrared light and a visible light emitted from a target.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

References will be made to embodiments of the present invention, examples of which may be illustrated in the accompanying figures. These figures are intended to be illustrative, not limiting. Although the present invention is generally described in the context of these embodiments, it should be understood that it is not intended to limit the scope of the present invention to these particular embodiments.

Figure (“FIG”) 1 schematically illustrates an example ecosystem for medical imaging, in accordance with embodiments of the present disclosure.

FIG. 2 schematically illustrates a system for medical imaging in accordance with in accordance with embodiments of the present disclosure.

FIG. 3 illustrates a partially exploded side view of a first medical imaging apparatus in accordance with embodiments of the present disclosure.

FIG. 4 illustrates a side view of a coupler in accordance with embodiments of the present disclosure.

FIG. 5 illustrates a perspective exploded view of a coupler in accordance with embodiments of the present disclosure.

FIG. 6 illustrates a partially exploded perspective view of a first sterile drape assembly aligned with a first medical imaging apparatus in accordance with embodiments of the present disclosure.

FIG. 7 illustrates a partially exploded side view of a first sterile drape assembly aligned with a first medical imaging apparatus in accordance with embodiments of the present disclosure.

FIG. 8 illustrates a side view of an apparatus for explaining an optical path of light in the apparatus where a first medical imaging apparatus and a first sterile drape assembly are coupled with each other in accordance with embodiments of the present disclosure.

FIG. 9 illustrates a perspective view of a second sterile drape assembly in accordance with embodiments of the present disclosure.

FIG. 10 illustrates a longitudinal cross-sectional view taken along line L-L of FIG. 9 showing the interior details of the second sterile drape assembly in accordance with embodiments of the present disclosure.

FIG. 11 illustrates a perspective exploded view of a sterile adapter aligned with an optical connector and a camera in accordance with embodiments of the present disclosure.

FIG. 12 illustrates a perspective view of an apparatus where a second medical imaging apparatus and a second sterile drape assembly are coupled with each other in accordance with embodiments of the present disclosure.

FIG. 13 illustrates an exploded side view of an apparatus for explaining an alignment of a camera, an optical connector and a second sterile drape assembly prior to connection in accordance with embodiments of the present disclosure.

FIG. 14 illustrates a side view of an apparatus for explaining an optical path of light in the apparatus where a second medical imaging apparatus and a second sterile drape assembly are coupled with each other in accordance with embodiments of the present disclosure.

FIG. 15 illustrates a partially exploded side view of a third medical imaging apparatus in accordance with embodiments of the present disclosure.

FIG. 16 illustrates a partially exploded perspective view of a third sterile drape assembly aligned with a third medical imaging apparatus in accordance with embodiments of the present disclosure.

FIG. 17 illustrates a side view of an apparatus for explaining an optical path of light in the apparatus where a third medical imaging apparatus and a third sterile drape assembly are coupled with each other in accordance with embodiments of the present disclosure.

FIG. 18 schematically illustrates an example flowchart of a method for medical imaging in accordance with embodiments of the present disclosure.

FIGS. 19A to 19K illustrate comparative images of a tissue site obtained by a subject apparatus for medical imaging in accordance with embodiments of the present disclosure.

FIGS. 20 and 21 schematically illustrate a machine learning algorithm that is operatively coupled to the subject system for medical imaging in accordance with embodiments of the present disclosure.

FIG. 22 schematically illustrates a computer system that is programmed or otherwise configured to implement methods provided herein.

FIG. 23 illustrates a simplified block diagram an exemplary computer node that can be used in connection with the medical imaging apparatus disclosed herein.

### DETAILED DESCRIPTION OF PREFERRED EMBODIMENTS

In the following description, for purposes of explanation, specific details are set forth in order to provide an understanding of the invention. It will be apparent, however, to one skilled in the art that the invention can be practiced without these details. Furthermore, one skilled in the art will recognize that embodiments of the present invention, described below, may be implemented in a variety of ways, such as a process, an apparatus, a system, a device, or a method using the system, the device or the apparatus.

Components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components that may be implemented in software, hardware, or a combination thereof. Furthermore, connections between components within the figures are not intended to be limited to direct connections. Rather, data between these components may be modified, re-formatted, or otherwise changed by intermediary components or devices. Also, additional or fewer connections may be used.

It shall also be noted that the terms “coupled” “connected” or “communicatively coupled” shall be understood to comprise direct connections, indirect connections through one or more intermediary devices, and wireless and/or wired connections.

Furthermore, by applying relevant technology, one skilled in the art shall recognize: (1) that certain steps may optionally be performed; (2) that steps may not be limited to the specific order set forth herein; (3) that certain steps may be performed in different orders; and (4) certain steps may be done concurrently.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments.

In the following description, it shall also be noted that the terms “learning” shall be understood not to intend mental action such as human educational activity of referring to performing machine learning by a processing module such as a processor, a CPU, an application processor, micro-controller, and so on.

A “feature(s)” is defined as a group of one or more descriptive characteristics of subjects that can discriminate for a tissue. A feature can be a numeric attribute.

The terms “comprise/include” used throughout the description and the claims and modifications thereof are not intended to exclude other technical features, additions, components, or operations.

Unless the context clearly indicates otherwise, the singular forms “a,” “an,” and “the” may be intended to include the plural forms as well. Also, when description related to a known configuration or function is deemed to render the present disclosure ambiguous, the corresponding description is omitted.

FIG. 1 schematically illustrates an example ecosystem for medical imaging, in accordance with embodiments of the present disclosure. The ecosystem may comprise a target site of a subject (e.g., a tissue site of interest of a patient). The ecosystem may comprise a medical imaging apparatus 200. The ecosystem may comprise a light source unit 400b in optically communication with the medical imaging apparatus 200. The light source unit 400b may be configured to provide one or more light beams (e.g., a combined light beam) via the medical imaging apparatus 200 and toward the target 100. The target 100 may be in optically communication with the medical imaging apparatus 200, such that the target 100 may be illuminated by the one or more light beams from the medical imaging apparatus 200 and the medical imaging apparatus 200 may detect one or more light signals reflected or emitted by the target 100 upon such illumination. The medical imaging apparatus 200 may be configured to capture at least one image or video of the target based on at least a portion of the one or more light signals from the target 100. A camera control unit 400a may be configured to analyze or combine data, image(s), or video(s) generated by the medical imaging apparatus 200. A computing unit 400c may communicate with the camera control unit 400a, and extract feature information from data, image(s), video(s) received from the camera control unit 400a based on a machine learning model installed therein.

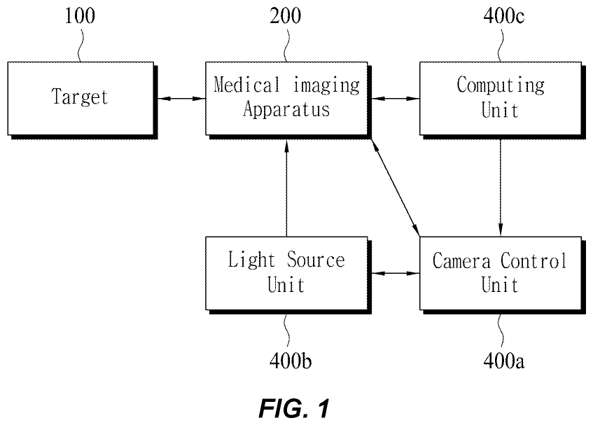

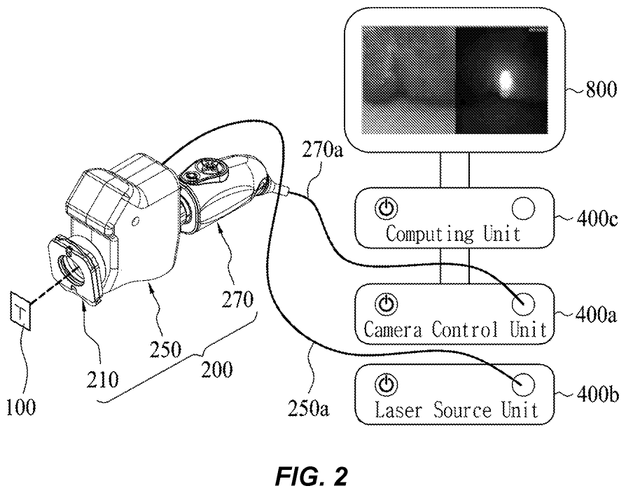

FIG. 2 schematically illustrates a system for medical imaging in accordance with in accordance with embodiments of the present disclosure.

Referring to FIG. 2, The system may comprise the medical imaging apparatus 200, a light source unit 400b, a computing unit 400c, a camera control unit 400a and a display unit 800. The medical imaging apparatus 200 may comprise a coupler 210, an optical connector 250 and a camera 270 or may consist of the optical connector 250 and the camera 270. The coupler 210 may comprise one side configured to releasably couple to a sterile drape (not shown) that protects the medical imaging apparatus 200 from external contamination, and another side configured to releasably couple to the optical connector 250. Also, the coupler 210 may comprise one side configured to releasably couple to a scope assembly (not shown) and another side configured to releasably couple to the optical connector 250. The optical connector 250 may comprise an optics assembly therein, one portion configured to optically couple to the light source unit 400b through an optical transmission means 250a such as an optical cable, and another portion configured to releasably or optically couple to the camera 270. The camera 270 may comprise an image sensor for sensing light emitted from a target (e.g., a patient's tissue) therein, and may be couple to a signal transmitting means 270a for transmitting a light signal sensed by the image sensor to the camera control unit 400a. The image sensor may be configured in the camera 270 to receive the light signal from the target of the subject for analysis and/or visualization of the target of the subject. Such light signal may be reflected or emitted from the target. The image sensor may be configured to detect the light signal from the target and transform the detected light signal to generate an image indicative of the target tissue. The generated image may be one-dimensional or multidimensional (e.g., two-dimensional, three-dimensional, etc.). Alternatively, the image sensor may be operatively coupled to a processor. In such case, the image sensor may be configured to detect the light signal from the target and convert the detected light signal into a digital signal. The image sensor may further be configured to transmit the digital signal to the processor that is capable of generating an image indicative of the target tissue. Examples of the image sensor may include, but are not limited to, a charge coupled device (CCD), metal oxide semiconductor (MOS) (e.g., complementary MOS, i.e., CMOS), modifications thereof, functional variants thereof, and modifications thereof. The camera control unit 400a may comprise an image processor used for image processing for the light signal obtained from the camera 270 therein. The light source unit 400b may comprise a light source having one or more different wavelengths (e.g., 785 nm or 830 nm) to provide light to the target 100 through the optical connector 250. The computing unit 400c may comprise a processor for extracting feature information from an image data based on a machine learning model for the image data processed by the camera control unit 400a. The display unit 800 may visualize the image data processed by the image processor of the camera control unit 400a, and may also visualize feature information extracted from the image data in the computing unit 400c.

The medical imaging apparatus 200 of the present disclosure may be usable for a number of medical applications, e.g., general surgery, neurosurgical procedures, orthopedic procedures, and spinal procedures. The medical imaging apparatus 200 of the present disclosure may be applicable to a wide variety of endoscopy-based procedures, including, but are not limited to, cholecystectomy, hysterectomy, thyroidectomy, and gastrectomy. In embodiments, the medical imaging apparatus 200 may be configured to be operatively coupled to a scope assembly for medical imaging. The medical imaging apparatus 200 may enhance one or more functions (e.g., imaging functions) of the scope assembly. The medical imaging apparatus 200 may introduce one or more additional functions (e.g., imaging functions) to the scope assembly. The medical imaging apparatus 200 may allow a user (e.g., a medical practitioner such as a physician, nurse practitioner, nurse, imaging specialist, etc.) to visualize and/or analyze a target of a subject, such as internal tissue of a patient, in one or more ways that any traditional scope assembly alone cannot.

A portion (e.g., a coupler) of the medical imaging apparatus 200 may be reused, and may be interchangeable with different scope assemblies. The scope assembly may be configured to visualize external and/or inner surface of a tissue (e.g., skin or internal organ) of a subject. The scope assembly may be used to examine (e.g., visually examine) the tissue of the subject and diagnose and/or assist in a medical intervention (e.g., treatments, such as a surgery). In some cases, the scope assembly may be an endoscope. Examples of the endoscope may include, but are not limited to, a cystoscope (bladder), nephroscope (kidney), bronchoscope (bronchus), arthroscope (joints) and colonoscope (colon), and laparoscope (abdomen or pelvis).

The medical imaging apparatus 200 may be configured to receive one or more light signals from the target of the subject. The medical imaging apparatus 200 may be configured to receive at least one or more light signals from the target 100. The one or more light signals may be reflected or emitted from the target upon exposure or illumination of the target to an optical beam.

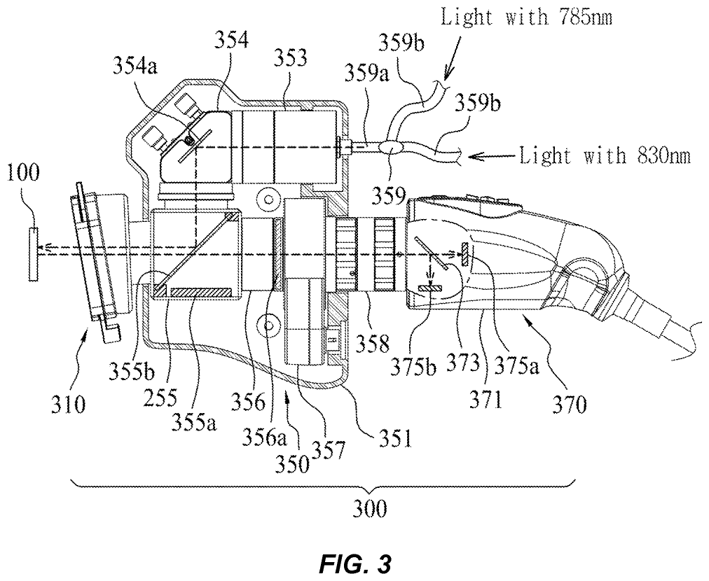

FIG. 3 illustrates a partially exploded side view of a first medical imaging apparatus in accordance with embodiments of the present disclosure.

Referring to FIG. 3, a first medical imaging apparatus 300 may comprise a coupler 310, an optical connector 350 and a camera 370. the optical connector 350 may comprise a housing 351. The housing 351 may include one or more biologically acceptable and/or compatible materials suitable for medical applications, depending on the particular application and/or preference of a medical practitioner. For example, components of the housing may include or be fabricated from materials such as polyvinyl chloride, polyvinylidene chloride, low density polyethylene, linear low density polyethylene, polyisobutene, poly(ethylene-vinylacetate) copolymer, lightweight aluminum foil and combinations thereof, stainless steel alloys, commercially pure titanium, titanium alloys, silver alloys, copper alloys, Grade 5 titanium, superelastic titanium alloys, cobalt-chrome alloys, stainless steel alloys, superelastic metallic alloys (e.g., Nitinol, super elasto-plastic metals, ceramics and composites thereof such as calcium phosphate, thermoplastics such as polyaryletherketone (PAEK) including polyetheretherketone (PEEK), polyetherketoneketone (PEKK) and polyetherketone (PEK), carbon-PEEK composites, PEEKBaS04 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, glass, and combinations thereof.

At least a portion of the housing 351 may be opaque, semi-transparent, or transparent. In some cases, the housing 351 may be opaque and configured to block any external light from entering through the housing 351 into one or more components within the housing 351 and interfering with the one or more light signals from the target of the subject that is received by the optical connector 350.

Pressure inside the housing 351 of the optical connector 350 may be approximately the same as ambient pressure (e.g., atmospheric pressure). Alternatively, the pressure inside the housing 351 may be controlled (or regulated, e.g., manually or automatically) such that the inner pressure of the housing 351 is lower or higher than the ambient pressure. Temperature inside the housing 351 of the optical connector 350 may be approximately the same as ambient temperature (e.g., room temperature). Alternatively, the temperature inside the housing 351 may be controlled (or regulated, e.g., manually or automatically) such that the inner temperature of the housing 351 is lower or higher than the ambient temperature. Humidity inside the housing 351 of the optical connector 350 may be approximately the same as ambient humidity. Alternatively, the humidity inside the housing 351 may be controlled (or regulated, e.g., manually or automatically) such that the inner humidity of the housing 351 is lower or higher than the ambient humidity. In some examples, the pressure, temperature, and/or humidity of the optical connector 350 may be regulated for optimal function of the optical connector 350.

The housing 351 may comprise an optics assembly disposed in the housing 351. The optics assembly may be configured to receive light signals that are reflected from a target 100 within a subject's body and transmitted through the coupler 310. Examples of the target 100 within the subject's body can include, but are not limited to, thyroid gland, adrenal gland, mammary gland, prostate gland, testicle, trachea, superior vena cava, interior vena cava, lung, liver, gallbladder, kidney, ureter, appendix, bladder, urethra, heart, esophagus, diaphragm, aorta, spleen, stomach, pancreas, small intestine, large intestine, rectum, vagina, ovary, bone, thymus, skin, adipose, brain, fetus, arteries, veins, nerves, ureter, bile duct, healthy tissue, diseased tissue and different types of tumor such as, adrenal gland tumor and thyroid tumor.

The optics assembly may comprise various components such as, for example, a first body tube 353 in which a lens for collimating incident external light is disposed, a first casing 354 in which a kinematic mirror 354a is mounted, a second casing 355 having a beam dump 355a and a beam splitter 355b such as a single-wavelength notch laser dichroic beamsplitter or a multi-wavelengths notch laser dichroic beamsplitter disposed therein, a second body tube 356 for forming an optical path and having a single or multi-wavelength notch filter 356a therein, a third casing 357 having an electrically tunable lens disposed therein, and a third body tube 358 having a fixed focal length lens disposed therein. In embodiments, the beam dump 355a may be located on the opposite portion of the surface of the beam splitter 355b on which the light is incident in the second casing 355 in order to absorb a beam of light that has partially passed through the beam splitter 355b without being reflected from the beam splitter 355b. The beam dump has almost no reflectivity, and its materials include certain types of acrylic paint, carbon nanotubes, anodized aluminum, and nickel-phosphate coatings.

The optics assembly is not limited to the above components, but may replace any optical means capable of performing their function, such as a polarizer instead of the single-wavelength or multi-wavelengths notch laser dichroic beamsplitter. An optic coupler 359 may be optically connected to the first body tube 353 through an optical cable 359a so that each light transmitted through optical fibers 359b is independently incident into the optical connector 350. In this case, the wavelength of each light incident along the optical fibers 359b may be ranged between 750 nm and 785 nm or 830 nm, but may be a unique wavelength band in which autofluorescence can be generated from each target.

The camera 370 may comprise a case 371. The case 371 may comprise a beam splitter 373 optically disposed therein to separate light incident from the optical connector 350 according to wavelength of the light, and first and second sensors 375a, 375b for sensing the separated light. The first sensor 375a may sense near-infrared light, and the second sensor 375b may sense visible light.

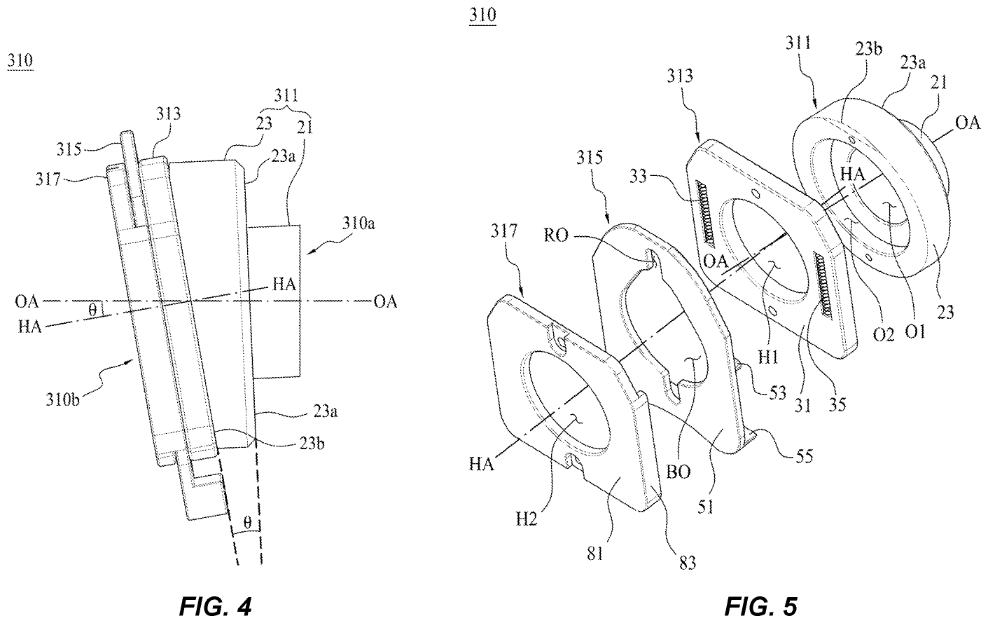

FIG. 4 illustrates a side view of a coupler in accordance with embodiments of the present disclosure, FIG. 5 illustrates a perspective exploded view of a coupler in accordance with embodiments of the present disclosure.

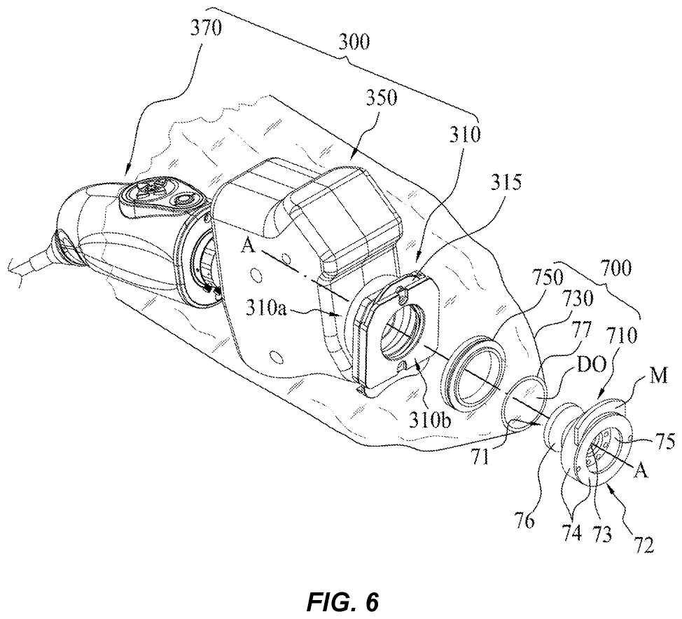

Referring to FIGS. 4 and 5, the coupler 310 may comprise an adapting unit 311, a connecting unit 313, a securing unit 315 and a covering unit 317, which are serially disposed along an optical path in which light reflected from the target 100 travels toward the camera 370, as described in FIG. 3. In embodiments, the coupler 310 includes a first end 310a which attaches to the optical connector 350, and a second end 310b which engages to a sterile adapter 710 shown in FIG. 6.

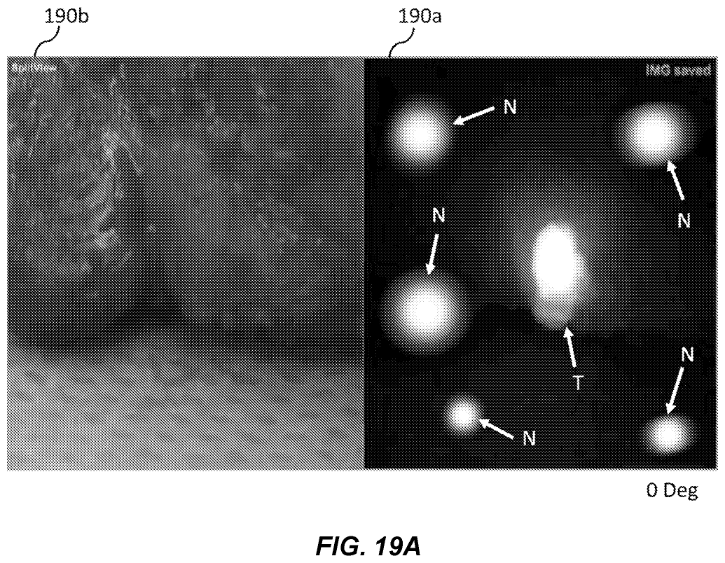

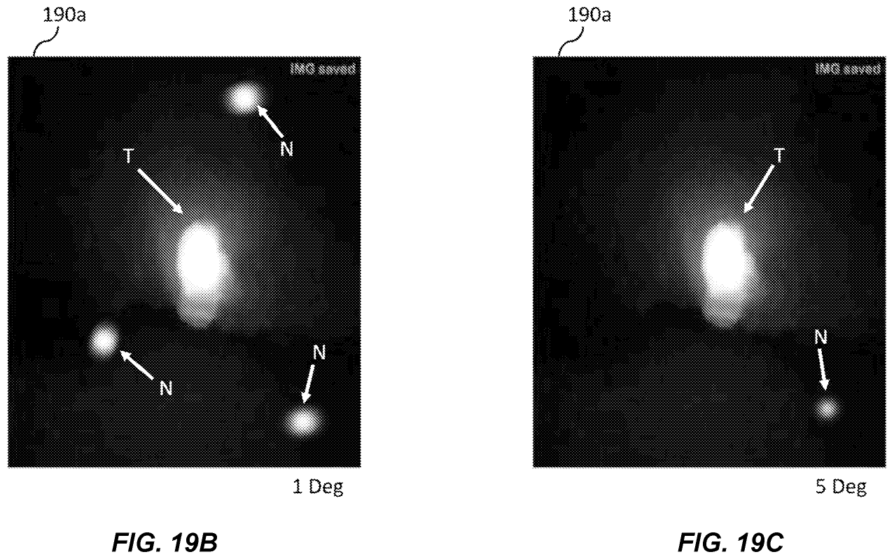

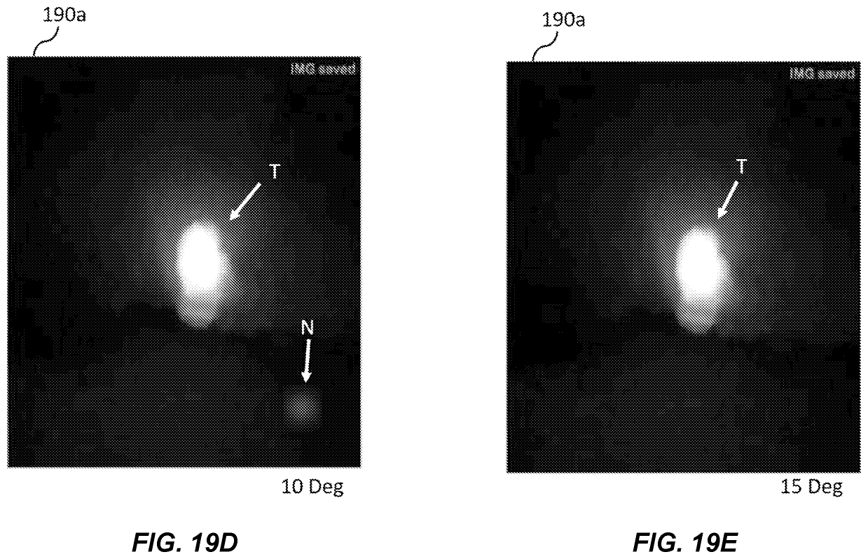

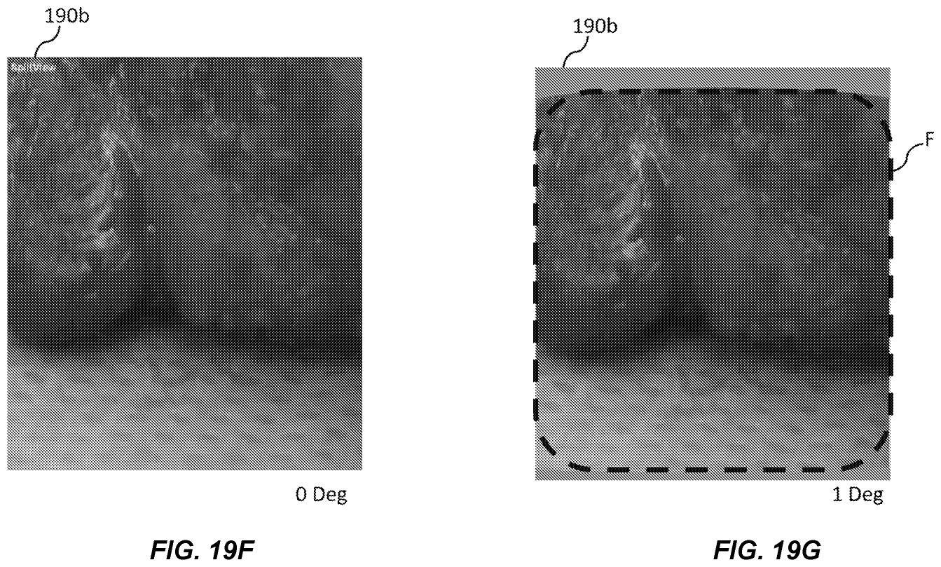

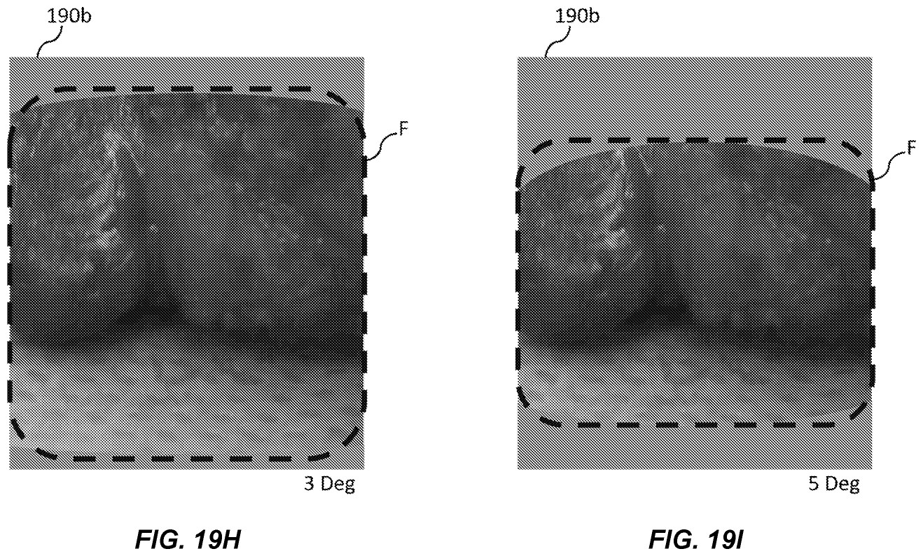

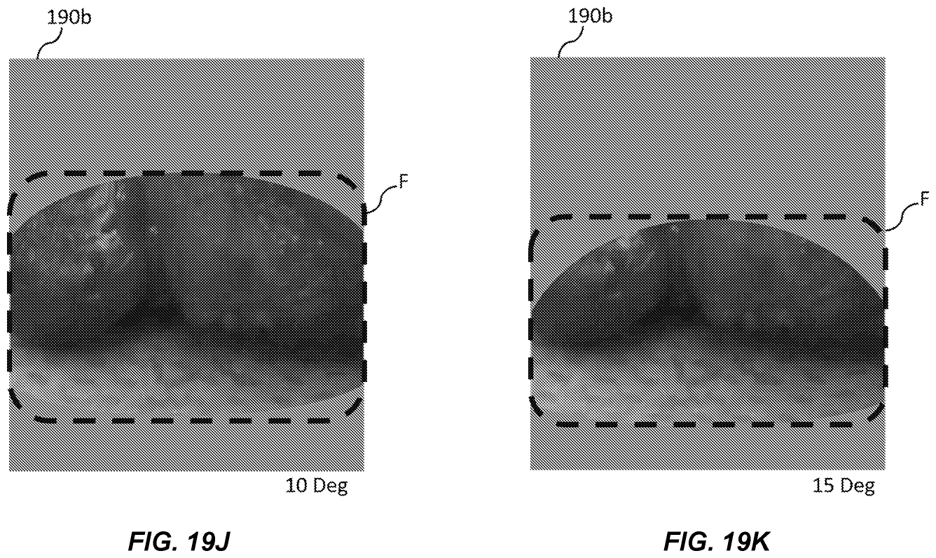

In embodiments, the adapting unit 311 may include a fixing part 21 having a first opening O1 for passing the light and an angle adjustment part 23 having a second opening O2 in spatially communication with the first opening O1. The diameter of the second opening O2 is larger than the diameter of the first opening O1. The angle adjustment part 23 may be configured to be integral with the fixing part 21. Each of the fixing part 21 and the angle adjustment part 23 may be formed in an annular shape. One side 23a of the angle adjustment part 23 attached to the fixing part 21 is perpendicular to a central axis OA-OA of the first opening O1 and the second opening O2, and the opposite side 23b of the angle adjustment part 23 may be inclined with a predetermined angle θ with respect to the one side 23a of the angle adjustment part 23. The predetermined angle may range 1 degree or more. As describe in FIGS. 19A to 19K below, considering a field of view of the camera 370, the predetermined angle preferably may range from about 1 to 15 degrees. More preferably, the predetermined angle may range from about 3 to 15 degrees.

In embodiments, the connecting unit 313 may be configured to couple to the adapting unit 311. The connecting unit 313 may include a plate 31 having a circular hollow H1 corresponding to the second opening O2 in the center of the plate 31, and an insertion groove 33 formed around the circular hollow H1. A spring member 35 disposed within the insertion groove 33 may the securing unit 315 with spring tension to releasably engage the sterile adapter 710.

In embodiments, the securing unit 315 may include a baseplate 51 having a barrel-shaped opening (i.e., a barrel-shaped hollow) BO with rectangular openings RO extending from the top and bottom of the barrel-shaped opening BO, a guide protrusion 53 protruding from one side of the baseplate 51 to insert into the insertion groove 33 of the connecting unit 313, and a grip plate 55 extending parallel to the guide protrusion 53 from an edge portion of the baseplate 51. The securing unit 315 may be a sliding quick release mechanism which is similar to the mount portion M of the sterile adapter 710, as described in conjunction with FIG. 6. The quick release mechanism may be configured to releasably couple the coupler 310 to various types of sterile adapter 710 having different sizes. In an example, the second portion 310b of the coupler 310 may comprise different sections with varied dimensions (e.g., different radial dimensions) configured to releasably coupled to different sterile adapter 710 having different sizes. In another example, the second portion 310b may comprise an adjustable aperture mechanism with adjustable aperture diameter to accommodate different the sterile adapter 710 having different sizes. The quick release mechanism may be configured to quickly move between a lock position (i.e., a coupled position) and a release position (i.e., a non-coupled position) in response to one or more movements of the quick release mechanism, such as a single, non-repetitious movement (e.g., lateral or rotational) of the quick release mechanism. The quick release mechanism may be configured to quickly move between a lock and a release position in response to a user instruction via a switch. The quick release mechanism may allow for precise coupling of two members, such as the second portion 310b of the coupler 310 and the sterile adapter 710. The precise coupling may provide an optimal optical path between the two members. The quick release mechanism may be configured to permit the user to releasably couple the second portion 310b of the coupler 310 to the sterile adapter 710 without use of tools. Alternatively, the quick release mechanism may be configured to permit the user to releasably couple the second portion 310b of the coupler 310 to the sterile adapter 710 with one or more tools, e.g., one or more keys to operatively coupled to the quick release mechanism to activate release of the quick release mechanism. The quick release mechanism may be configured to permit the user to releasably couple the second portion 310b of the coupler 310 to the sterile adapter 710 in less than 30 seconds. In some cases, the coupling between the second portion of 310b the coupler 310 and the sterile adapter 710 may not utilize a quick release mechanism. In this case, the sterile adapter 710 may be screwed on to the second portion of the coupler 310, thereby preventing a quick release of the sterile adapter 710 from the second portion 310b of the coupler 310. In an example, a coupling surface of the second portion 310b of the coupler 310 may substantially mimic the structure of a coupling surface of the sterile adapter 710.

In embodiments, the covering unit 317 may include a cover plate 81 having a circular hollow H2 corresponding to the circular hollow H1 of the connecting unit 313 and a flange 83 extending perpendicular to the surface of the cover plate 81 at an edge portion of the cover plate 81 so that the flange is coupled to cover the securing unit 315 and the connecting unit 313.

Thus, the overall structure of the coupler 310 may be formed so that the central axis HA of the barrel-shaped opening BO of the securing unit 315 and the central axis HA of the circular hollows H1, H2 of the connecting unit 313 and the covering unit 317 are coaxial, and coaxial axis HA of the barrel-shaped opening BO and the circular hollows H1, H2 is tilted with a predetermined angle θ with respect to a central axis OA of the openings O1, O2 of the adapting unit 331.

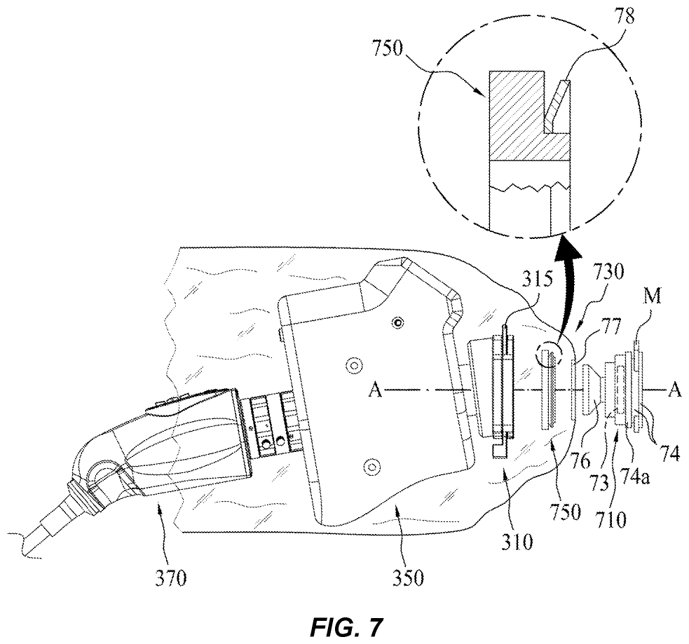

FIG. 6 illustrates a partially exploded perspective view of a first sterile drape assembly 700 aligned with a first medical imaging apparatus 300 in accordance with embodiments of the present disclosure, FIG. 7 illustrates a partially exploded side view of the first sterile drape assembly 700 aligned with the first medical imaging apparatus 300 in accordance with embodiments of the present disclosure.

Referring to FIGS. 6 and 7, a medical imaging system of the present disclosure may comprise the first medical imaging apparatus 300 and the first sterile drape assembly 700. A coupler 310, an optical connector 350, a camera 370 constituting the first medical imaging apparatus 300 and a sterile drape 730, a sterile adapter 710 constituting the first sterile drape assembly 700 are aligned along with an optical axis A-A. The coupler 310 of the first medical imaging apparatus 300 may comprise a first portion 310a in which light is incident from the optical connector 350, and a second portion 310b in which the light passes through an inside of the coupler 310 and is emitted. The first portion 310a may be coupled to the optical connector 350 and the second portion 310b may be coupled to the sterile adapter 710. The first sterile drape assembly 700 may comprise a sterile adapter 710, a drape 730 and a locking ring 750. The sterile adapter 710 may be configured substantially similar to the coupler 310. The sterile adapter 710 includes a first end 71 which attaches to the coupler 310, and a second end 72 which may connect or disconnect to an endoscope (not shown). In embodiments, a window 73 may be positioned within the sterile adapter 710 for providing a sterile barrier between the coupler 310 and the endoscope. The sterile adapter 710 may comprise an endoscope mount 74 having an interior passageway 75 which optically communicates with the window 73. Furthermore, the sterile adapter 710 may comprise an optical coupler mount 76 also including an interior passageway which communicates with the opposite side of the window 73. In order to attach the sterile adapter 710 to a desired endoscope, the sterile adapter 710 may include a securing means such as sliding quick release mechanism M which is similar to the securing unit 315 of the coupler 310. The Mechanism M is mounted transversely with respect to axis A-A which defines the longitudinal direction. In operation, a sliding quick release M may be depressed to enlarge the opening within interior passageway enabling an endoscope to be inserted therein. Upon relieving pressure on the M, a portion of quick release M then engages an eyepiece of the endoscope. A spring member (not shown) disposed within endoscope mount 74 provides the sliding quick release M with spring tension to releasably engage the endoscope. In embodiments, the drape 730 may include a drape ring 77 which may be constructed of a circular shaped wire that is integral with the distal end of the drape 730. The drape ring 77 defines a drape opening DO. The sterile adapter 710 with respect to the first medical imaging apparatus 300 including the coupler 310 and the locking ring 750 may be aligned as shown in FIG. 6, when the first sterile drape assembly 700 and the first medical imaging apparatus 300 of the present embodiments is in use. However, prior to use, the sterile adapter 710 and the drape 730 may be packaged such that the drape 730 extends over the second end 72 of the sterile adapter 710 with the locking ring 750 exposed exteriorly of the drape 730. Thus, it will be understood that when in use, the drape 730 is inverted so that it is pulled back over the first end 71 of the sterile adapter 710 and completely over the coupler 310, the optical connector 350, the camera 370 and its trailing cables. Referring to FIG. 7, the locking ring 750 may include sealing means such as spring washer 78 to provide a water and airtight seal for capturing the drape 730 therebetween. More specifically, the endoscope mount 74 may include an engagement flange 74a which receives drape ring 77. The contact of washer 78 against the drape ring 77 which is pressed against the engagement flange 74a ensures a tight seal. In lieu of the spring washer 78, a Teflon® seal may be used to provide the liquid and airtight seal.

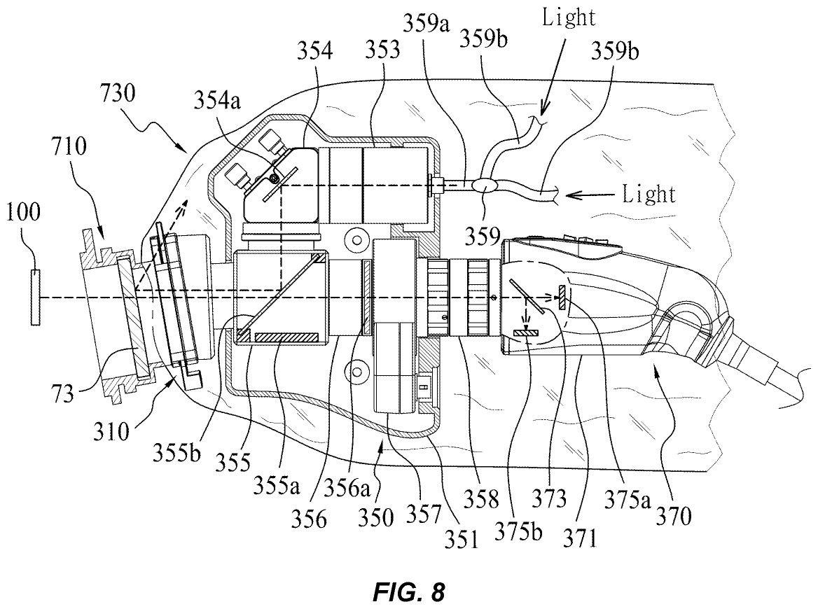

FIG. 8 illustrates an optical path of light in an apparatus where the first medical imaging apparatus and the first sterile drape assembly are coupled with each other in accordance with embodiments of the present disclosure.

Referring to FIG. 8, the light of any wavelength (e.g., 785 nm or 830 nm) input into an optical connector 350 through an optical cable passes through optical components such as a kinematic mirror 354a, a beam splitter 355b included therein and is incident on a sterile adapter 710 coupled to a coupler 310. At this time, the light passes through a window 73 located in the sterile adapter 710 and is incident on a target 100, and some light in is reflected from a surface of the window 73. A reflection of light at the surface of the window 73 is not a reflection that can adversely affect the visualization of the image of the target 100. That is, Retroreflection does not occur at the surface of the window 73 because a first portion 310a of the coupler 310 is tilted to a second portion 310b of the coupler 310 so that the incident light is not perpendicular to the surface of the window 73, as described in FIG. 6. Thus, as shown in FIGS. 19A to 19K below, when the image is visualized by the image sensors 375a, 375b disposed in a camera 370 with light emitted from the target 100, the present apparatus enables the coupler 310 to remove noise such as white spots that can be locally generated by light reflection in the visualized NIR image for identifying the target (e.g., a tissue), thereby preventing misunderstanding and confusion in recognizing the target 100 by medical practitioners. On the other hand, when light having any wavelengths (e.g., 785 nm or 830 nm) is incident on the tilted window 73, some light is reflected in the direction of the camera 370 and may generate a noise in visualizing the image of the target (e.g., a parathyroid gland or an adrenal gland tumor), as shown in FIGS. 19B to 19D. In case of the present apparatus, the light reflected from the window 73 can be optically filtered by the beam splitter 355b within the optical connector, and the light passing unfiltered in the beam splitter 355b can be completely filtered by the notch filter 356a placed in the optical path of the passing light, thereby removing the noise in the visualized image.

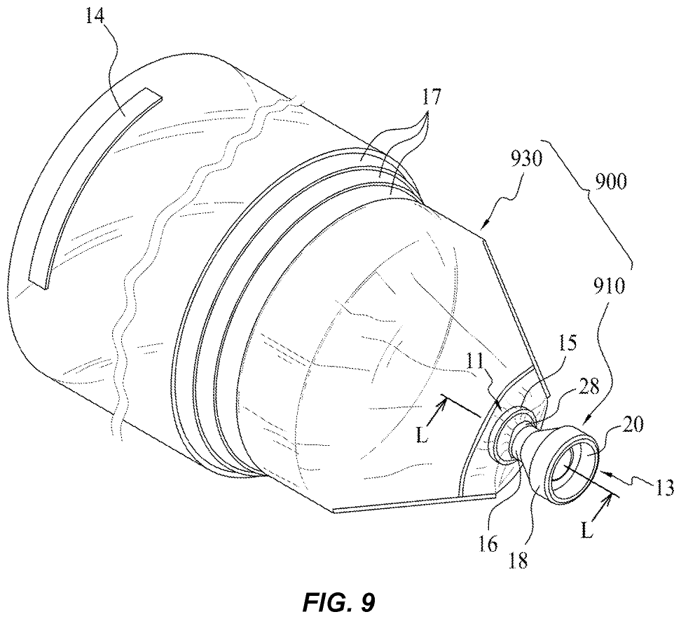

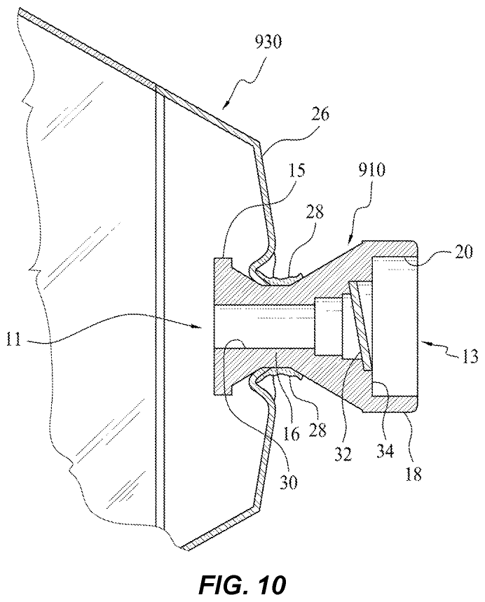

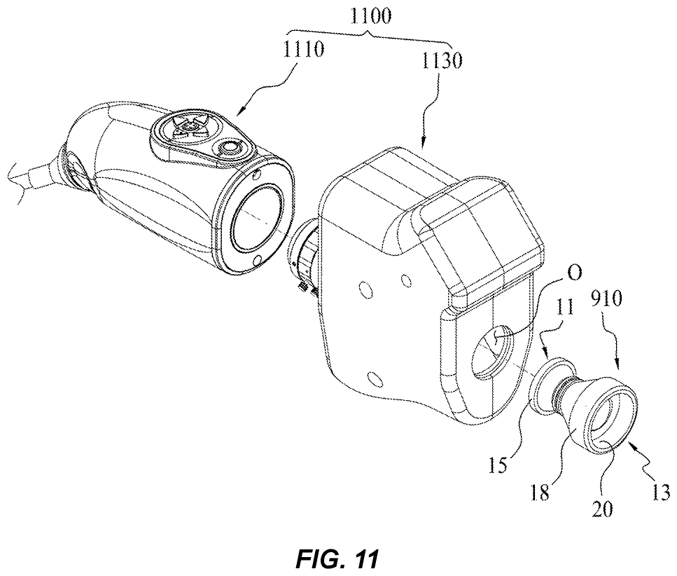

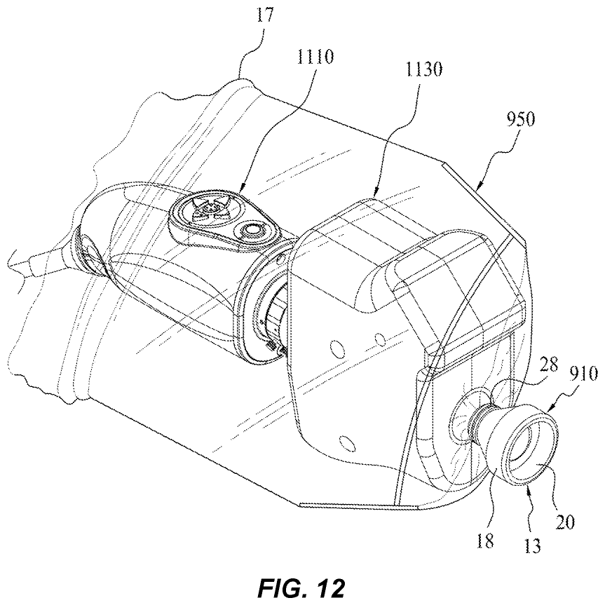

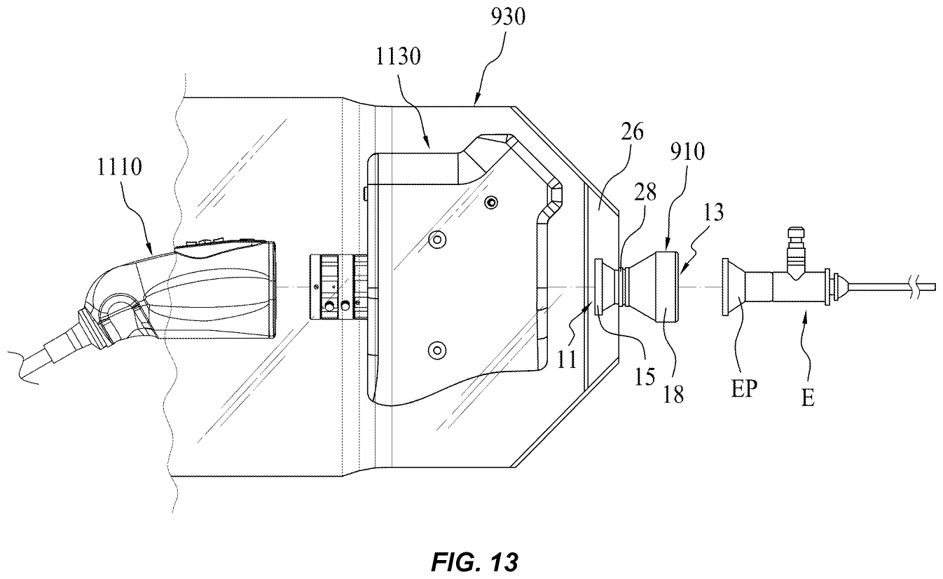

FIG. 9 illustrates a perspective view of a second sterile drape assembly in accordance with embodiments of the present disclosure, FIG. 10 illustrates a longitudinal cross-sectional view taken along line L-L of FIG. 9 showing the interior details of the second sterile drape assembly in accordance with embodiments of the present disclosure, FIG. 11 illustrates a perspective exploded view of a sterile adapter aligned with an optical connector and a camera in accordance with embodiments of the present disclosure, FIG. 12 illustrates a perspective view of an apparatus where a second medical imaging apparatus and a second sterile drape assembly are coupled with each other in accordance with embodiments of the present disclosure, FIG. 13 illustrates an exploded side view of an apparatus for explaining an alignment of a camera, an optical connector and a second sterile drape assembly prior to connection in accordance with embodiments of the present disclosure.

Referring to FIGS. 9 to 13, the second sterile drape assembly 900 may comprise a disposable sterile adapter 910 and a disposable drape 930, and be provided for coupling a second medical imaging apparatus 1100 comprising an unsterile video camera 1110 and unsterile optical connector 1130 to a sterile endoscope or not. The overall structure of the sterile adapter 910 and drape 930 can best be seen by viewing FIGS. 9 and 10. The sterile adapter 910 may include a first end 11 having an annular mounting 15 which attaches to a common optical connector such as a “C” mount connector. This annular mounting 15 may resemble the eyepiece of a standard endoscope. The sterile adapter 910 further may include a second end 13 having an endoscope mount 18 characterized by a substantially cylindrical shape which may be configured to match the particular type of endoscope used. In embodiments, the endoscope mount 18 may include an interior cylindrical wall 20 for receiving a standard endoscope having an annular eyepiece, as described in conjunction with FIG. 13. A neck portion 16 may be disposed between the annular mounting 15 and endoscope mount 18. An annular mounting 15 may be inserted within an opening located at the distal end 26 of the sterile drape 930 such that the opening surrounds the neck portion 16. A sealing means 28 such as surgical tape may be used to seal the distal end 26 against the neck portion 16, thus providing a sterile curtain wall between the first and second ends of the adapter 910. Sealing and bonding of the sterile drape 930 to the sterile adapter 910 may also be done by a variety of methods, including adhesives, shrink-wrap or double-faced adhesive strips. The sterile drape 930 may include folds 17 in order to reduce the size of the drape for storage prior to use. As shown in FIG. 9, the folds 17 may be telescopic wherein consecutive drape sections are folded on top of one another, or alternatively the folds 17 may be folded in a roll configuration (not shown) like a condom. If the folds 17 are telescopic, pull tab 14 may be provided in order to extend the drape for use. As seen in FIG. 10, the primary purpose of sterile adapter 910 is to provide an optical pathway and sterile barrier between a sterile endoscope E and a second medical imaging apparatus 1100 including video camera 1110 and optical connector 1130 described in conjunction with FIG. 13. Accordingly, the interior passageway 30 is provided to allow light to be transmitted from the sterile endoscope to the video camera 1110. To maintain sterility, optically clear window 32 may be located on the optical pathway of the sterile adapter 910 and be provided which allows the passage of light. Also, the window 32 may provide a sterile barrier between the video camera 1110 and sterile endoscope that may be coupled to the sterile adapter 910. The window 32 may be tilted to ends 11, 13 of sterile adapter 910 so that the incident light from the optical connector 1130 is not perpendicular to the surface of the window 32. As best seen in FIGS. 10 and 13, for use of the sterile adapter 910 with an endoscope E of the type having a conventional eyepiece EP, the eyepiece EP is inserted within endoscope mount 18 such that the eyepiece EP is pressed flush against interior wall 34 and window 32. Retaining screws (not shown) may be connected with endoscope mount 18 and may then be used to secure the eyepiece EP. Alternatively, endoscope mount 18 could be configured like securing unit 315 of the coupler 310 or mount portion M of sterile adapter 710 shown in FIGS. 4 to 8, in order to receive the standard eyepiece EP of an endoscope. Other methods of securing the mount 18 to the endoscope are possible within the intended scope of this disclosure. The endoscope mount 18 may be configured to match any member of differing types of endoscopes.

The sterile adapter 910 may be made of a suitable plastic or metal material which is sterilizable by various methods such as gas sterilization or gamma radiation and thus is made completely sterile. A suitable material for the coupler may be polycarbonite or PETG, or possibly acrylic or styrene. Similarly, the sterile drape 930 may be made out of a material such as polyethylene preferably from 1 to 6 mils in thickness, that is sterilizable also making the drape completely sterile.

In embodiments, as shown in FIGS. 11 to 13, a video camera 1110 and an optical connector 1130 are inserted within the proximal end of the sterile drape 930. Opening portion O of the optical connector 1130 is coupled to annular mounting 15 of the sterile adapter 910. The video camera 1110 may then be attached to the optical connector 1130. The sterile drape 930 may be then pulled back over the optical connector 1130, the video camera 1110 and its trailing cables thus providing a sterile covering isolating the medical imaging apparatus from the sterile operating environment. The sterile endoscope E may then be coupled with the endoscope mount 18 of the sterile adapter 910. The sterile endoscope E may be secured by appropriate securing mean such as retaining screws. If it is desired to use a different type of endoscope having differing optical qualities, retaining screws are simply released and the sterile endoscope E is removed from the endoscope mount 18. A new endoscope may then be introduced wherein sterility is maintained during the change in endoscopes. After use, the optical connector 1130 and the endoscope E are disconnected from the sterile adapter 910, and the sterile drape 930 and the sterile adapter 910 are thrown away.

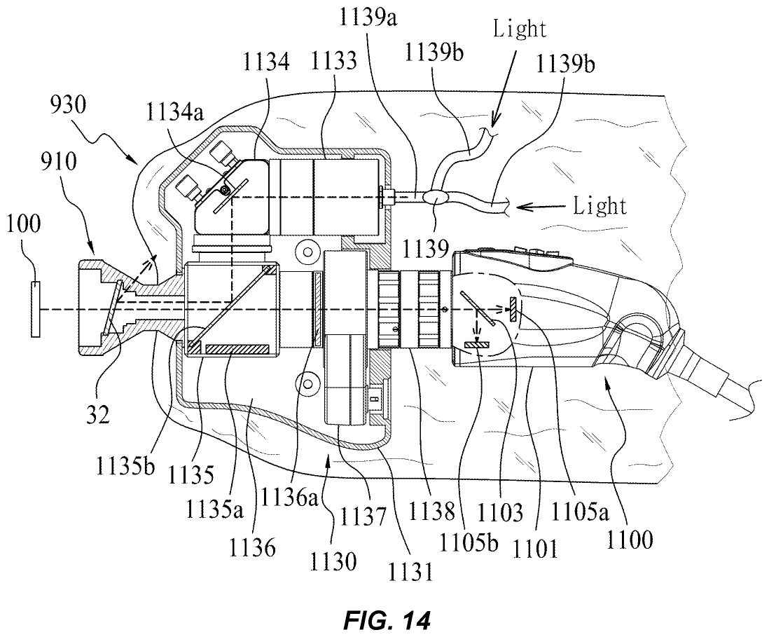

Referring to FIG. 14, the light input into an optical connector 1130 through an optical cable passes through optical components such as a kinematic mirror 1134a, a beam splitter 1135b and is incident on a sterile adapter 910 directly coupled to optical connector 1130. At this time, the light passes through a window 32 located within the sterile adapter 910 and is incident on a target 100, and some light in is reflected from a surface of the window 32. A reflection of light at the surface of the window 32 is not a reflection that can adversely affect the visualization of the image of the target 100. That is, Retroreflection does not occur at the surface of the window 32 because of the sterile adapter 910 designed so that the incident light is not perpendicular to the surface of the window 32. Thus, as shown in FIGS. 19A to 19K below, when the image is visualized by the image sensors disposed in a camera 1110 with light emitted from the target 100, the present apparatus enables sterile adapter 910 to remove noise such as white spots that can be locally generated by light reflection in the visualized NIR image, thereby preventing misunderstanding and confusion in recognizing the target 100 by medical practitioners. Meanwhile, when light having any wavelengths (e.g., 785 nm or 830 nm) is incident on the tilted window 32, some light is reflected in the direction of the camera 1100 and may generate a noise in visualizing the image of the target (e.g., a parathyroid gland or an adrenal gland tumor), as shown in FIGS. 19B to 19D. In case of the present apparatus, the light reflected from the window 32 can be optically filtered by the beam splitter 1135b within the optical connector, and the light passing unfiltered in the beam splitter 1135b can be completely filtered by the notch filter 1136a placed in the optical path of the passing light, thereby removing the noise in the visualized image.

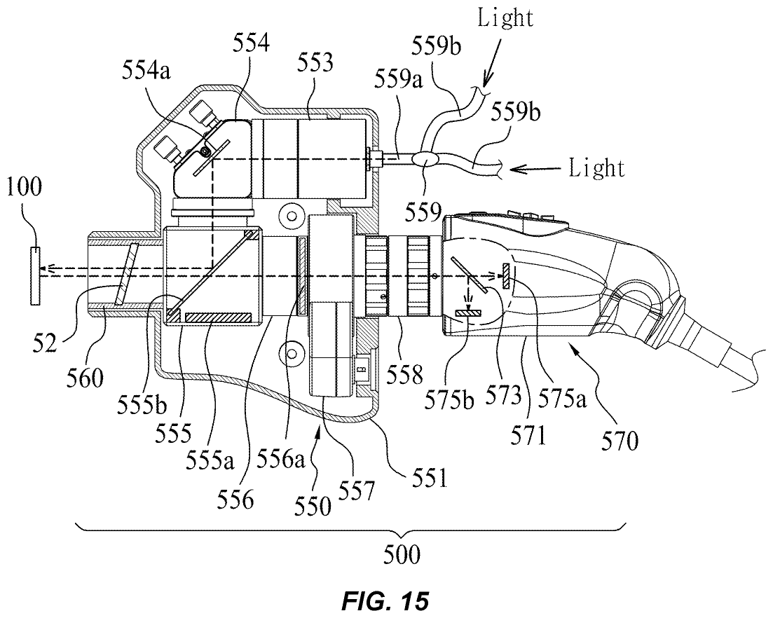

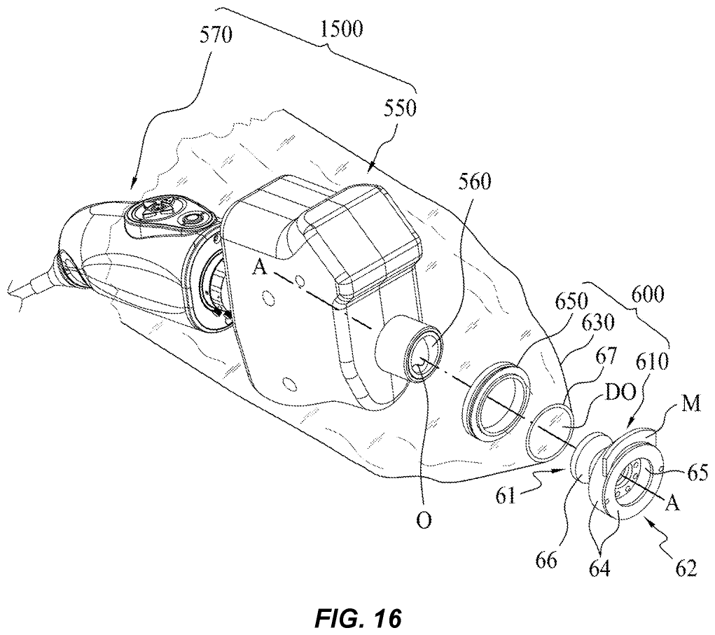

FIG. 15 illustrates a partially exploded perspective view of a third sterile drape assembly aligned with a third medical imaging apparatus in accordance with embodiments of the present disclosure, FIG. 16 illustrates a partially exploded side view of a third medical imaging apparatus in accordance with embodiments of the present disclosure.

Referring to FIGS. 15 and 16, the components of third medical imaging apparatus 1500 may be formed similarly to their counterpart of the first medical imaging apparatus 300 except for the coupler 310 shown in FIG. 3. The optics assembly included to the housing 551 may further comprise a fourth body tube 560 for forming an optical path in which the reflected light from a beam splitter 555b travels toward a target 100. A window 52 may be disposed within the fourth body 560 tube to allow the passage of light. Also, the window 52 may be tilted to an end 51 of the fourth body tube 560 so that the incident light from the beam splitter 555b is not perpendicular to the surface of the window 52. The components of third sterile drape assembly 600 may be configured substantially similar to their counterpart of the first sterile drape assembly 700 described in FIG. 6. A sterile adapter 610 of the third sterile drape assembly 600 includes a first end 61 which attaches to an opening portion O of the optical connector 550, and a second end 62 which may connect or disconnect to an endoscope (not shown). In embodiments, the sterile adapter 610 may be spatially in communication with the fourth body tube 560 so that an optical path is formed without any component therein, such as the window 73 in FIGS. 6 and 7.

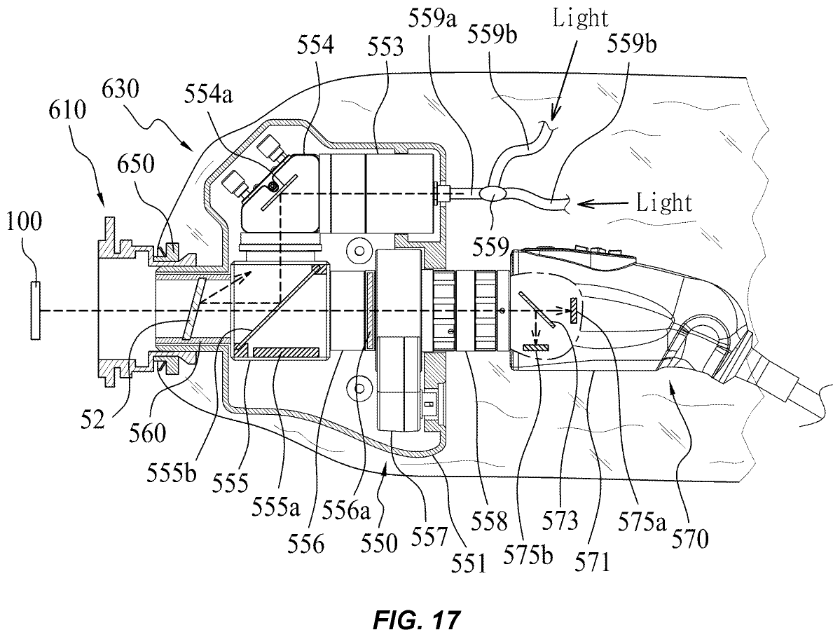

FIG. 17 illustrates a side view of an apparatus for explaining an optical path of light in the apparatus where a third medical imaging apparatus and a third sterile drape assembly are coupled with each other in accordance with embodiments of the present disclosure.

Referring to FIG. 17, the light input into an optical connector 550 through an optical cable passes through optical components such as a kinematic mirror 554a, a beam splitter 555b and then is incident toward a fourth body tube 560. At this time, the light passes through a window 52 located within the fourth body tube 560 and is incident on a target 100, and some light in is reflected from a surface of the window 52. A reflection of light at the surface of the window 52 is not a reflection that can adversely affect the visualization of the image of the target 100. That is, Retroreflection does not occur at the surface of the window 52 because of the optical connector 550 designed so that the incident light is not perpendicular to the surface of the window 52. Thus, as shown in FIGS. 19A to 19K below, when the image is visualized by the image sensors disposed in a camera 570 with light emitted from the target 100, the present apparatus enables optical connector 550 to remove noise such as white spots that can be locally generated by light reflection in the visualized NIR image, thereby preventing misunderstanding and confusion in recognizing the target 100 by medical practitioners. Meanwhile, when light having any wavelengths (e.g., 785 nm or 830 nm) is incident on the tilted window 52, some light is reflected in the direction of the camera 570 and may generate a noise in visualizing the image of the target 100 (e.g., a parathyroid gland or an adrenal gland tumor), as shown in FIGS. 19B to 19D. In case of the present apparatus, the light reflected from the window 52 can be optically filtered by the beam splitter 555b within the optical connector, and the light passing unfiltered in the beam splitter 555b can be completely filtered by the notch filter 556a placed in the optical path of the passing light, thereby removing the noise in the visualized image.

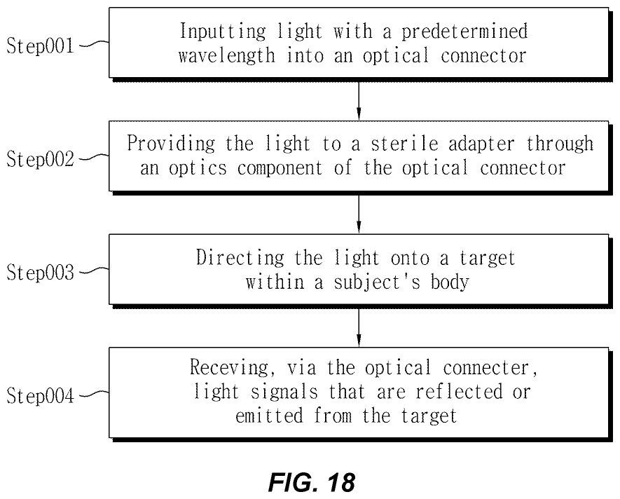

Any subject medical imaging apparatus of the present disclosure can be used for medical imaging of a target of a subject. In an aspect, the present disclosure provides a method of using an apparatus that may include a medical imaging apparatus and a sterile drape assembly for medical imaging. FIG. 18 schematically illustrates an example flowchart of a method for medical imaging in accordance with embodiments of the present disclosure. The method may comprise providing a medical imaging apparatus and a sterile drape assembly including a sterile adapter and a drape comprising. The method may comprise inputting light with a predetermined wavelength into an optical connector. In some cases, the predetermined wavelength of light ranges from 785 nm to 830 nm (Step 001). The method may further comprise providing the light to the sterile adapter through an optics assembly of optical connector (Step 002). The method may further comprise directing the light onto a target within the subject's body (Step 003). The method may further comprise receiving, via the optical connector, light signals that are reflected or emitted from the target (Step 004). Alternatively, the method may comprise receiving, via an additional optical path, the light signals that are reflected or emitted from the target. In some cases, the additional optical path may be formed by the coupler. the coupler may be disposed between the sterile adapter and the optical connector when releasably coupled thereto. The coupler, the optical connector, and the camera may share a common longitudinal axis.

Any one of the subject medica imaging apparatus of the present disclosure may be used to visualize anatomy, morphology, one or more physiological features, and/or one or more pathological features of a target within a subject's body.

FIGS. 19A to 19K illustrate comparative images of a tissue site obtained by a subject apparatus for medical imaging in accordance with embodiments of the present disclosure. Referring to FIG. 19A, when one end of the coupler 310 included in the medical imaging apparatus 300 is tilted by 0 degree with respect to the surface of the window 73 included in the sterile adapter 710, as described in FIG. 8, the subject's tissue (e.g., a thyroid including parathyroid gland) is imaged by the medical imaging apparatus 300 as a NIR image 190a and a RGB image 190b. The NIR image 190a and the RGB image 190b are paired with each other. Although the parathyroid tissue T is observed in the NIR image 190a obtained by irradiating the subject's tissue with near-infrared rays, it can be seen that noise such as white spots is generated around the parathyroid tissue T as the near-infrared rays are reflected from the window as described above. Referring to FIGS. 19B to 19E, it can be seen that the number of noises N appearing in the NIR image 190a is changed according to the angle (e.g., 1, 3, 5, 10, 15 degrees) at which one end of the coupler is inclined with respect to the surface of the window increases, the noise disappears in the NIR image 190a. Accordingly, the present medical imaging apparatus enables practitioners to clearly identify the location of the tissue when imaging the tissue.

Referring to FIGS. 19F to 19K, as one end of the coupler 310 included in the medical imaging apparatus 300 is inclined with a predetermined angle (e.g., 1, 3, 5, 10, 15 degrees) with respect to the surface of the window 73, the field of view F of the camera 370 is changed when the subject's tissue is imaged as the RGB images 190b by the medical imaging apparatus 300. That is, it can be seen that the camera's field of view F narrows according to the angle at which one end of the coupler 310 is inclined with respect to the surface of the window 73 increases.

In some embodiments, the medical imaging apparatus of the present disclosure may be operatively coupled to a processor (e.g., a computer) configured to analyze a light signal data set (e.g., light spectra, images, or videos) captured by the medical imaging apparatus and identify a type of tissue or one or more features thereof. In an example, the medical imaging apparatus may use hyperspectral imaging to identify the tissue type. The processor may be capable of employing one or more machine learning algorithms to analyze a database comprising a plurality of known or previously collected data sets (e.g., light spectra, images, or videos) related to a plurality of tissue or features thereof. The one or more machine learning algorithms may be capable of analyzing the light signal data set from the image sensor of the camera. The one or more machine learning algorithms may comprise an artificial neural network. The artificial neural network may involve a network of simple processing elements (i.e., artificial neurons) which can exhibit complex global behavior, determined by the connections between the processing elements and element parameters. With or without a training set (e.g., database of previously identified tissue sites and features thereof along with respective light signal data sets), the artificial neural network may enhance the analysis capability of the machine learning algorithms.

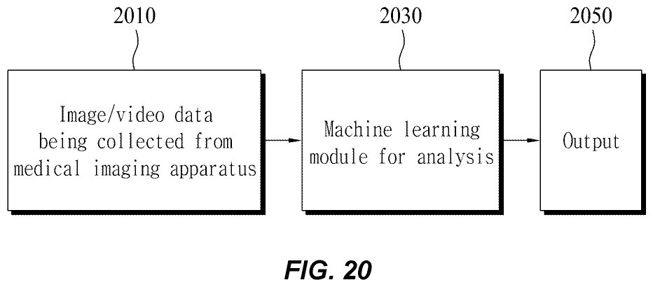

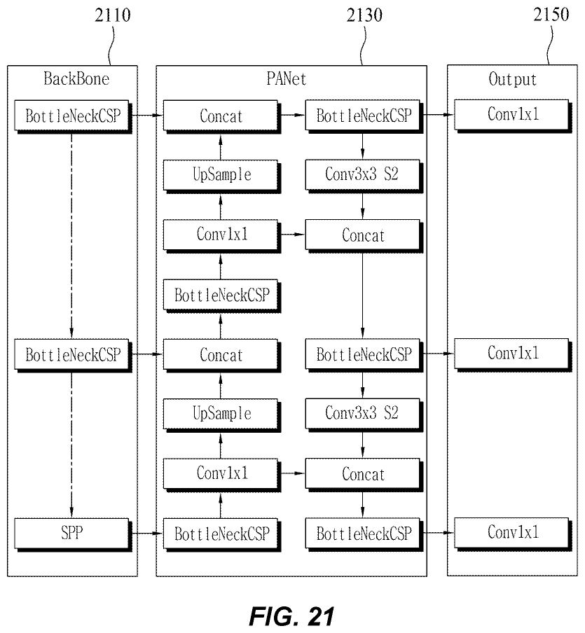

FIGS. 20 and 21 schematically illustrate a machine learning algorithm that is operatively coupled to the subject system for medical imaging in accordance with embodiments of the present disclosure. As shown in FIG. 20, the one or more machine learning algorithms of the present disclosure may comprise: (i) an input 2010 comprising image/video data that is collected from at least the medical imaging apparatus of the present disclosure, (ii) a machine learning module 2030 for analysis of the input 2010, and (iii) an output 2050. As shown in FIG. 21, the artificial neural network of the one or more machine learning algorithms, for example, YOLO, may comprise an input layer (Backbone) 2110, one or more hidden layers (PANet) 2130 including at least two hidden layers, and an output layer (Output) 2150. The one or more hidden layers may take in input signals (e.g., the light signal data), analyze them, and convert them into an output (e.g., an identified tissue type). In some cases, first, the data is inputted to CSPDarknet for feature extraction. It is then fed to PANet for feature fusion. Lastly, the YOLO Layer outputs detection results, such as the class, score, location, and size. Faster R-CNN was also used to compare the performance with YOLO. Faster R-CNN object (e.g., a tissue) detection network is composed of a feature extraction network followed by a region proposal network to generate object proposals and another subnetwork to predict the actual class of each object proposal. The feature extraction network may be typically a pretrained CNN, such as ResNet-50 or Inception v3.

Meanwhile, in order to solve the problem of limited size of training data, the machine learning may be used a technique called transfer learning, where a model's structure and parameter trained for one task is reused as the starting point for another task. The neural networks may be initialized by the pretrained weights on the dataset. The dataset may be collected not only as clean images but also as low-quality, blurry images from videos to make a model robust to motion artifacts during surgery. Also, data augmentation may be used to improve network accuracy by randomly transforming the original data during training. It shall be noted that data augmentation is not applied to test and validation data. To take the images of low resolution in real-time situations into account, the data may be augmented by blurring the image with the probability of 0.1. To evaluate the trained object detector on a large set of images to measure the performance, Mean Average Precision (mAP) at different intersection over union (IoU) levels (mAP.5:.95) may be used as an evaluation metric for object (e.g., a tissue) detection. A general definition of the AP (Average Precision) may be calculating the area under the precision-recall curve. The precision-recall curve may be created by calculating precisions at each level of confidence threshold. The AP may provide a single number that incorporates the ability of the detector to make correct classifications (precision) and the ability of the detector to find all relevant objects (recall). For example, mAP.5:.95 means average AP over different IOU thresholds, from 0.5 to 0.95 with a step size of 0.05.

In some cases, the light signal data input may comprise at least wavelength (e.g., more than 3 wavelengths, up to 1000 wavelengths, etc.). In some cases, the output layer 2150 may comprise one or more members of the following: (i) tissue identification utilizing NIR image data and/or RGB image data, (ii) spatial location (e.g., X, Y, Z Cartesian coordinates) of the tissue or features thereof, (iii) surgical decision support (e.g., proceed vs. abort), (iv) geofencing of critical structures within the tissue of interest.

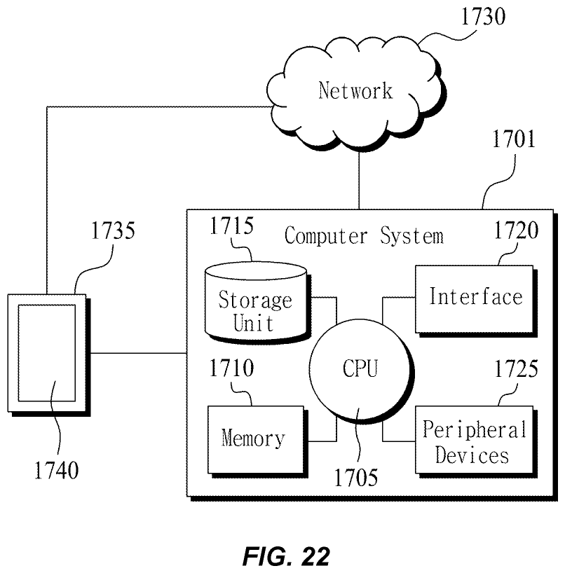

FIG. 22 shows a computer system that is programmed or otherwise configured to implement a method for medical imaging.

Referring to FIG. 22, the present disclosure provides the computer system 1701 that are programmed or otherwise configured to implement methods of the disclosure, e.g., any of the subject methods for medical imaging. The computer system 1701 may be configured to, for example, direct an illumination source and direct an image sensor of a camera to receive at least a portion of a light signal that is reflected or emitted by a target of a subject upon illumination by light beam. The computer system 1701 may be an electronic device of a user or a computer system that is remotely located with respect to the electronic device. The electronic device can be a mobile electronic device.

The computer system 1701 may comprise a central processing unit (CPU, also “processor” and “computer pro-processor” herein) 1705, which can be a single core or multi core processor, or a plurality of processors for parallel processing. The computer system 1701 also comprises memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are communicatively coupled to the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can comprise one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can comprise fetch, decode, execute, and writeback.

The CPU 1705 can be part of a circuit, such as an integrated circuit. One or more other components of the system 1701 can be comprised in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can comprise one or more additional data storage units that are located external to the computer system 1701 (e.g., on a remote server that is in communication with the computer system 1701 through an intranet or the Internet).

The computer system 1701 can communicate with one or more remote computer systems through the network 1730. For instance, the computer system 1701 can communicate with a remote computer system of a user (e.g., a subject, an end user, a consumer, a healthcare provider, an imaging technician, etc.). Examples of remote computer systems comprise personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can comprise any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements comprises optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media comprise dynamic memory, such as main memory of such a computer platform. Tangible transmission media comprise coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore comprise for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can comprise or be in communication with an electronic display 1735 that comprises a user interface (UI) 1740 for providing, for example, a portal for a healthcare provider or an imaging technician to monitor or track one or more features of the optical connector (e.g., coupling to the scope, coupling to the camera, the image sensor, the optics assembly, etc.). The portal may be provided through an application programming interface (API). A user or entity can also interact with various elements in the portal via the UI. Examples of UFs comprise, without limitation, a graphical user interface (GUI) and web-based user interface.

Methods and systems of the present disclosure can be implemented by way of one or more algorithms. An algorithm can be implemented by way of software upon execution by the central processing unit 1705.

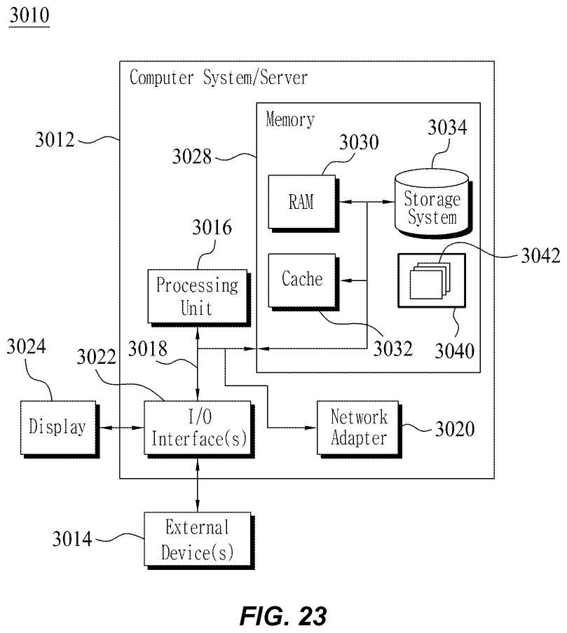

FIG. 23 illustrates a simplified block diagram an exemplary computer node that can be used in connection with the medical imaging apparatus disclosed herein.

Referring to FIG. 23, a schematic of an exemplary computing node is shown that may be used with the medical imaging systems described herein. A computing node 3010 is only one example of a suitable computing node and is not intended to suggest any limitation as to the scope of use or functionality of embodiments described herein. Regardless, the computing node 3010 may be capable of being implemented and/or performing any of the functionality set forth hereinabove.

The computing node 3010 may include a computer system/server 3012, which is operational with numerous other general purpose or special purpose computing system environments or configurations. Examples of well-known computing systems, environments, and/or configurations that may be suitable for use with computer system/server 3012 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system/server 3012 may be a camera control unit 400a or a computing unit 400c in FIG. 2 and be described in the general context of computer system-executable instructions, such as program modules, being executed by a computer system. Generally, program modules may include routines, programs, objects, components, logic, data structures, and so on that perform particular tasks or implement particular abstract data types. The computer system/server 3012 may be practiced in distributed cloud computing environments where tasks are performed by remote processing devices that are linked through a communications network. In a distributed cloud computing environment, program modules may be located in both local and remote computer system storage media including memory storage devices. As depicted in FIG. 23, the computer system/server 3012 in computing node 3010 is shown in the form of a general-purpose computing device. The components of computer system/server 3012 may include, but are not limited to, one or more processors or processing units 3016, a system memory 3028, and a bus 3018 coupling various system components including system memory 3028 to processor 3016.

Bus 3018 may comprise one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 3012 may include a variety of computer system readable media. Such media may be any available media that is accessible by computer system/server 3012, and may include both volatile and non-volatile media, removable and non-removable media.

System memory 3028 can include computer system readable media in the form of volatile memory, such as random access memory (RAM) 3030 and/or cache memory 3032. Computer system/server 3012 may further include other removable/non-removable, volatile/non-volatile computer system storage media. By way of example only, storage system 3034 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 3018 by one or more data media interfaces. As will be further depicted and described below, memory 3028 may include at least one program product having a set (e.g., at least one) of program modules that are configured to carry out the functions of embodiments of the disclosure.

Program/utility 3040, having a set (at least one) of program modules 3042, may be stored in memory 3028 by way of example, and not limitation, as well as an operating system, one or more application programs, other program modules, and program data. Each of the operating system, one or more application programs, other program modules, and program data or some combination thereof may include an implementation of a networking environment. Program modules 3042 generally carry out the functions and/or methodologies of embodiments described herein.

Computer system/server 3012 may also communicate with one or more external devices 3014 such as a keyboard, a pointing device, a display 3024, etc.; one or more devices that enable a user to interact with computer system/server 3012; and/or any devices (e.g., network card, modem, etc.) that enable computer system/server 3012 to communicate with one or more other computing devices. Such communication can occur via Input/Output (I/O) interfaces 3022. Still yet, computer system/server 3012 can communicate with one or more networks such as a local area network (LAN), a general wide area network (WAN), and/or a public network (e.g., the Internet) via network adapter 3020. As depicted, network adapter 3020 communicates with the other components of computer system/server 3012 via bus 3018. It should be understood that although not shown, other hardware and/or software components could be used in conjunction with computer system/server 3012. Examples, include, but are not limited to: microcode, device drivers, redundant processing units, external disk drive arrays, RAID systems, tape drives, and data archival storage systems, etc.

The present disclosure provides a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CDROM), a digital versatile disk (DVD), a memory stick, a floppy disk, a mechanically encoded device such as punchcards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In various embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In various alternative implementations, the junctions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A coupler for medical imaging comprising:
an adapting unit including an opening therein;
a connecting unit configured to couple to the adapting unit;
a securing unit configured to couple to the connecting unit; and
a covering unit configured to couple to the securing unit,
wherein each of the connecting unit, the securing unit and the covering unit has a hollow in spatially communication with the opening of the adapting unit and one side of the adapting unit is inclined with a predetermined angle with respect to the opposite side of the adapting unit.

2. The coupler of claim 1, wherein the adapting unit comprises a fixing part having a first opening for passing light and an angle adjustment part configured to be integral with the fixing part, having a second opening in spatially communication with the first opening of the fixing part.

3. The coupler of claim 2, wherein a diameter of the second opening is larger than the diameter of the first opening.

4. The coupler of claim 1, wherein the connecting unit comprises a plate with a groove and a spring member inserted into the groove.

5. The coupler of claim 1, wherein the securing unit comprises a baseplate, a guide protrusion protruding from one side of the baseplate, and a grip plate extending parallel to the guide protrusion from an edge portion of the baseplate.

6. The coupler of claim 1, wherein the covering unit comprises a cover plate and a flange extending substantially perpendicular to a surface of the cover plate.

7. The coupler of claim 1, wherein a central axis of the opening is tilted with a given angle with respect to a central axis of the hollow.

8. A medical imaging apparatus, comprising:
an optical connector;
a coupler configured to releasably couple to a first portion of the optical connector; and
a camera configured to releasably couple to a second portion of the optical connector,
wherein the coupler comprises a first portion in which light is incident from the optical connector, and a second portion in which the light passes through an inside of the coupler and is emitted,
wherein the first portion is tilted with a predetermined angle with respect to the second portion.

9. The medical imaging apparatus of claim 8, wherein a wavelength of the light ranges from 750 nm to 830 nm.

10. The medical imaging apparatus of claim 8, wherein the optical connector comprises a casing and a body tube disposed along an optical path of the light, wherein the casing includes a beam splitter and a beam dump located on an opposite portion of a surface of the beam splitter on which the light is incident in the casing, the body tube includes a notch filter for filtering the light.

11. The medical imaging apparatus of claim 8, wherein the camera comprises a first image sensor and a second image sensor for respectively sensing a near-infrared light and a visible light emitted from a target irradiated with the light.

12. The medical imaging apparatus of claim 8, wherein the second portion of the coupler is configured to releasably couple to a sterile adapter using a quick release mechanism.

13. The medical imaging apparatus of claim 8, wherein the coupler comprises an adapting unit including an opening therein; a connecting unit configured to couple to the adapting unit; a securing unit configured to couple to the connecting unit; and a covering unit configured to couple to the securing unit.

14. A medical imaging system, comprising:
an optical connector;
a sterile adapter; and
a coupler having a first portion to releasably couple to a first side of the optical connector and a second portion to releasably couple to the sterile adapter,
wherein the first portion of the coupler is tilted with a predetermined angle with respect to the second portion of the coupler.

15. The medical imaging system of claim 14, wherein the sterile adapter comprises a window positioned within the sterile adapter so that light incident from the coupler is not retroreflected at a surface of the window.

16. The medical imaging system of claim 15, wherein a wavelength of the light ranges from 750 nm to 830 nm.

17. The medical imaging system of claim 14, further comprising a sterile drape connected to the sterile adapter using a locking ring.

18. The medical imaging system of claim 14, further comprising a camera configured to releasably couple to a second side of the optical connector,
wherein the camera comprises a first image sensor and a second image sensor for respectively sensing a near-infrared light and a visible light emitted from a target.

19. The medical imaging system of claim 14, wherein the coupler comprises an adapting unit including an opening therein; a connecting unit configured to couple to the adapting unit; a securing unit configured to couple to the connecting unit; and a covering unit configured to couple to the securing unit.

20. The medical imaging system of claim 15, wherein the light is irradiated to a target passing through the window, and the light emitted from the target by the irradiated light passes through the window.
